(19) United States
(12) Patent Application Publication
(10) Pub. No.: US 2007/0191707 A1
(43) Pub. Date: Aug. 16, 2007

(54) Device for locating the cervix and uterus

(76) Inventor: Albert S. Denittis, Medford, NJ (US)

Correspondence Address: BLANK ROME LLP, 600 NEW HAMPSHIRE AVENUE, N.W., WASHINGTON, DC, 20037, US

(21) Appl. No.: 11/226,444

(22) Filed: Sep. 15, 2005

(51) Int. Cl. A61B 5/05

(52) U.S. Cl. 600/424

(57) ABSTRACT

A device and method of locating the cervix and uterus is disclosed. A means opaque to radiation is inserted through the cervix into the uterus and fixed to the cervix. A method of organ visualization may then be used to locate the position of the device and hence the cervix and uterus. The device may remain fixed in place inside the cervix and uterus for an extended period of time, allowing these organs to be easily located on a frequent basis.

### FIELD OF THE INVENTION

The present invention relates to a sleeve containing a marker opaque to radiation that allows for accurate visualization of the cervix and uterus with a variety of imaging techniques for the purpose of targeting radiation therapy.

### BACKGROUND OF THE INVENTION

Cervical and uterine cancers are widespread women's health problems. Uterine cancer is the most common cancer of the female reproductive system, and accounts for six percent of all of cancers in women in the United States. Cervical cancer is also a highly prominent disease. The American Cancer Society estimates that over 10,000 women in the United States will be diagnosed with cervical cancer in 2005. Although Pap screening tests have become more common, it is still estimated that more than 3,700 women die of cervical cancer each year in the United States alone. Treatment options for cervical and uterine cancer include surgery, chemotherapy and radiation therapy. Of these options, surgery is the most invasive, often requiring the removal of the cervix, uterus and surrounding reproductive organs. Radiation therapy combined with chemotherapy has been shown to be one of the most effective ways of treating cervical and uterine cancers. Radiation therapy of cervical and uterine tumors requires very high doses of radiation. Common current radiation therapy uses a vaginal and cervical implant to maximize radiation exposure to the cervix and uterus while minimizing the exposure of other tissues. This type of internal radiation treatment is intrusive, degrading and uncomfortable for women, and typically must be endured for 8 weeks. Because of these limitations of internal radiation, it is desirable to develop better methods for treating cervical and uterine cancers with external radiation.

External beam radiation therapy using x-rays has been used in cancer treatment for more than 65 years. Although this method is highly effective in stopping the proliferation of tumor cells, the technique is inaccurate, almost always causing damage to healthy tissue and debilitating side effects. The recently developed technique of Intensity Modulated Radiation Therapy (IMRT) allows for very precise radiation dosing to malignant tumors. IMRT uses three-dimensional images of the tumor and surrounding tissues to develop highly targeted radiation dosing (see Hong et al., British Journal of Cancer, 92:1819-1824(2005)). Because IMRT allows for targeted delivery to tumors, much higher doses of radiation may be used with a lower risk of side effects and damage to healthy tissue.

While IMRT is a highly targeted method of delivering radioactivity to a tumor, greater precision in the method is still desired. IMRT therapy is usually based on a computed tomography (CT) scan that visualizes the tumor and its location in the body. Often, this CT scan is taken before the beginning of treatment, and a day or more may lapse before the start of the treatment regimen. This is of special relevance because organs, such as the cervix and uterus, shift naturally inside the body, and their precise location varies from day to day. This movement of organs and the tumors associated with them decreases the accuracy of IMRT treatment, as that treatment is based on the CT scan or other image that was taken days or weeks before. Standard comprehensive imaging of the cervix before daily treatment is not often feasible, as the available imaging techniques often require the injection or consumption of contrast materials. Contrast materials are dyes or other compounds that are diluted in water or oil which make the linings of organs of the body more opaque to the type of imaging radiation being used. The administration of these contrast materials can take a considerable amount of time. Furthermore, the contrast materials can cause allergic reactions and discomfort in patients. A method that allows for rapid location of the cervix and uterus without the use of contrast materials would be of great utility to the medical community. Increased precision and targeting of the cervix and uterus will greatly improve the treatment regimen of cervical and uterine cancers, by allowing IMRT to be effectively and safely used in such treatments.

### SUMMARY OF THE INVENTION

It will be apparent from the above that the need exists for a device that allows for precise positioning of the cervix and uterus to allow for highly targeted external radiation therapy of cervical and uterine tumors. It is therefore an object of the present invention to provide a device that permits accurate visualization of the cervix and uterus during standard body imaging techniques. The device of this invention that can be used to accurately locate the cervix and uterus is a plastic sleeve comprising a marker opaque to radiation and, optionally, an inert filling material.

### DESCRIPTION OF THE DRAWINGS

A preferred but not limiting example of an embodiment of the invention is set forth in the drawings.

FIG. 1A is an elevated side view of an embodiment of the invention.

FIG. 1B is a cross-sectional view of the embodiment of FIG. 1A taken through line A.

FIG. 2 is a schematic showing how the embodiment of FIG. 1 can be fixed to the cervix inside the uterus.

### DETAILED DESCRIPTION OF THE INVENTION

The present invention is a device made up of a plastic sleeve with a marker insert that can be readily detected by standard body imaging techniques. The sleeve may then be sutured to the cervix, allowing for precise localization of the cervix and uterus during imaging.

A preferred embodiment of the device is set forth in the drawings. Throughout this disclosure, like numbers refer to like elements, with an increment of 100 between figures.

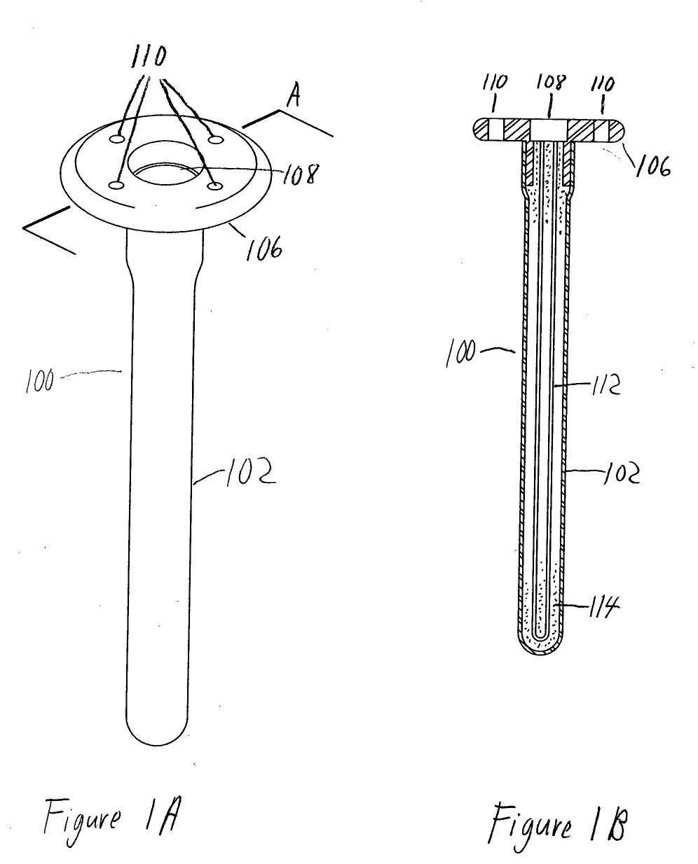

In a preferred embodiment, the device is modified Smit sleeve as shown in FIG. 1. As shown in FIG. 1A, the device 100 comprises a plastic sleeve 102 which is inserted through the cervix into the uterus. A circular endpiece 106 prevents overinsertion of the device. The circular endpiece 106 has a central hole 108 which allows for the introduction of materials into the sleeve, and suture holes 110 that allow for suturing of the device to the cervix. FIG. 1B shows a cross-sectional diagram of the device through line A of FIG. 1A. A metal solder wire 112 is introduced into the sleeve as the marker and held in place with an inert filling material 114. The metal solder wire can then be readily detected by standard body imaging techniques.

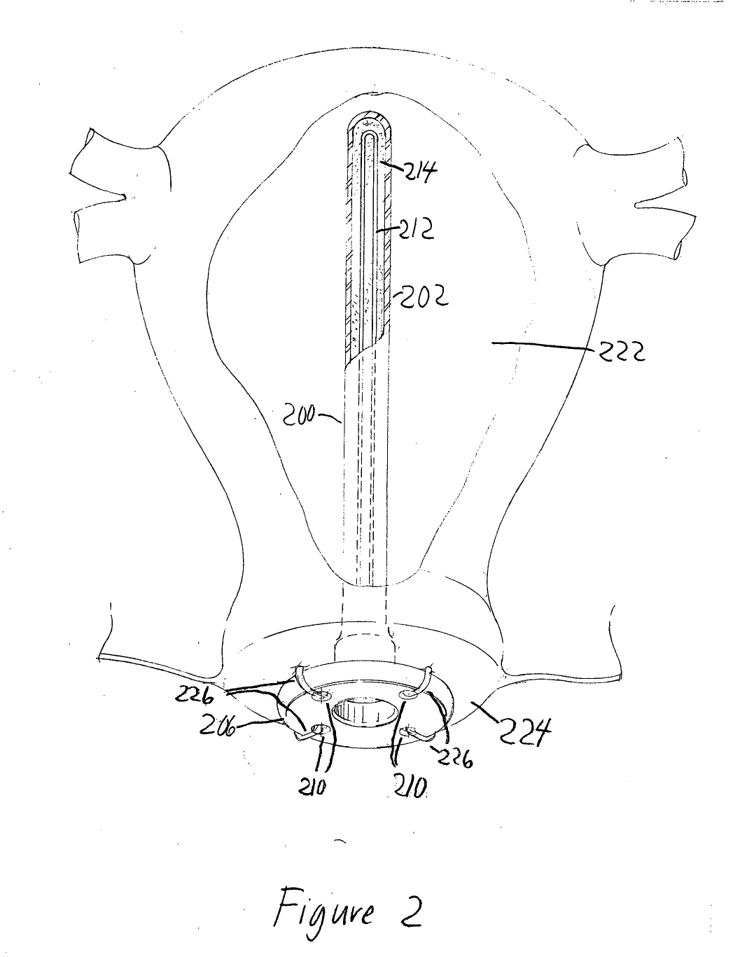

FIG. 2 shows an example of how the device shown in FIG. 1 can be fixed in the cervix. The device 200 is inserted through the cervical canal into the uterus 222 until the circular endpiece 206 comes in contact with the cervix 224. The device is then sutured to the cervix 224 using standard medical sutures 226 that run through the suture holes 210.

A Smit cervical sleeve or other suitable sleeve can be modified to form the present invention. In general use, a Smit sleeve is a cannula that can be sutured to the cervix to ease insertion of medical devices. In the present invention the Smit cervical sleeve has been modified and is no longer a cannula, but is filled with a marker that is detectable by a medical imaging technique and an inert material to hold the marker in place. The modification of the sleeve in no way prevents it from being attached, such as by use of sutures, to the cervix, where it will remain in place until removed. It will be apparent to one of skill in the art that various types of cannulae or other sleeves that could safely be inserted into and attached to the cervix could be used in an embodiment of the present invention.

In a preferred embodiment of the invention, the marker is made up of one or more pieces of metal of the same or various types. Non-limiting examples of metal pieces usable for the present invention are metal wires, cylinders and spheres. Further, the marker could be made of any material that is opaque to the types of radiation used in organ visualization techniques. The marker preferably should run the entire length of the sleeve to fill the entire uterus, but may also run only a fraction of the length of the sleeve. Several desirable qualities of the marker are necessary. Primarily, the marker needs to be readily detected by common organ visualization techniques. Also importantly, the marker must be able to be safely and permanently fixed within the plastic sleeve. Additionally, the marker must not have any properties which make it unsafe for the present invention to remain inside the cervix and uterus for extended periods of time, such as days or weeks. Those skilled in the art will recognize that this list of desirable properties for the marker is not necessarily limiting and that there are other properties of the marker that may or may not be present while still falling within the scope of the invention.

The inert filling material that fills the sleeve should be any material that safely holds the marker in place. A preferred example of inert filling material is vinyl polysiloxane, which is sold under the trade name Reprosil by Dentsply International, Inc. Reprosil is often used for making dental impressions and is described in U.S. Pat. Nos. 5,661,222, 5,863,965 and 6,561,807 by Hare, which are hereby incorporated by reference herein. This material is malleable and safe for use inside body cavities. The inert filling material must posses several qualities in order to be useable in the present invention. One important quality of the inert filling material is that it be able to fill the plastic sleeve in a manner that securely holds the marker in place while still allowing for the plastic sleeve to be attached to the cervix. The inert filling material must also not have any properties that would make it unsafe for the patient if it were to remain fixed inside the cervix and uterus for an extended period of time, such as days or weeks. It should be recognized that there are other properties that the inert filling material may or may not posses that could still allow for it to be useable in an embodiment of the present invention.

In an alternative embodiment of the present invention, the marker is held in place inside an unfilled or partially filled sleeve. The marker may be glued or fastened by mechanical means to remain held in place within the sleeve.

In a further embodiment of the invention, the device itself is made either entirely or partially from a material opaque to radiation. The device of this embodiment should posses many of the characteristics described above, mainly it must be able to be safely fixed inside the cervix and uterus for an extended period of time, such as days or weeks. It should be apparent to one of skill in the art that a device within the scope of the invention as claimed could be hollow or filled, and of various shapes and sizes. The primary characteristic of a device within the scope of the present invention is that it is opaque to radiation, either in part or in whole, and that it is able to be fixed in place inside the cervix and uterus so that the device, and hence the cervix and uterus, may be located using an organ visualization technique.

The device of the present invention must be able to be fastened in place to the cervix in a way that will not allow it to move or dislocate. As the device itself will be used for locating the cervix and uterus during imaging, it is of primary importance that the device must maintain a constant positioning relative to the position of the cervix and uterus for an extended period of time, such as days or weeks. In one embodiment of the present invention, the device is sutured to the cervix as shown in FIG. 2. Although the example in FIG. 2 involves the use of four sutures, it will be apparent to one of skill in the art that any number of sutures could be used in an embodiment of the present invention. Further, the device could comprise any number of suture holes, all of which may or may not be used by the surgeon attaching the device. Alternatively, another medical procedure or operative procedure could be used to fix the device to the cervix. Any procedure that can safely be performed on a patient to fix the device to the cervix could be used in an embodiment of the invention.

Techniques for Imaging the Body and Organs

A variety of organ imaging techniques are available to the medical practitioner, all of which are well known in the art. Examples of organ imaging techniques are discussed below. This list is not intended to be a comprehensive list of imaging techniques that are compatible with the instant invention, as other organ imaging techniques could be used in conjunction with the invention set forth here. A non-limiting description of techniques that can be used to visualize the cervix are summarized below and further described by Follen et al. (Cancer 98:2028-2038 (2003)) and Perez et al. (Rays 28:247-265 (2003)).

X-Ray Radiography

Traditional x-ray radiography can be used to visualize the cervix and uterus, by performing a hysterosalpingography, lymphangiograpy or similar procedure. Typically, visualization of the soft tissue structures of the female reproductive organs requires the administration of a contrast material, such as a water-soluble or oil-soluble dye. Standard 2-dimensional images of the pelvic region are then obtained and analyzed.

Computed Tomography

Computed tomography (CT scan or CAT scan) is a common x-ray technique that allows for 3-dimensional imaging of organs. A CT scan is made up of multiple x-ray pictures which are assembled and analyzed by computers to form a 3-dimensional picture of the area scanned. CT scans of the female reproductive organs typically require administration of contrast material to visualize the soft tissue structures.

Fluoroscopy

Fluoroscopy is another common x-ray technique that uses a continuous x-ray beam to take real-time images of the body. The results of a fluoroscopy scan are observed by the physician on a monitor while the technique is being administered. It is commonly used for the monitoring of bodily processes, such as the moving of the diaphragm, the flow of blood, or the movement of food through the digestive tract. Because it is an x-ray technique, fluoroscopy often requires the administration of contrast materials to visualize the soft tissue structures.

Magnetic Resonance Imaging

Magnetic resonance imaging (MRI) uses radiofrequency waves and a strong magnetic field instead of x-rays to image the organs and tissues of the body. MRI produces 3-dimensional images of the body by assembling multiple scans in a manner similar to CT scanning. MRI procedures can also require administration of contrast material and take longer to perform than CT scans.

Ultrasound

Ultrasound imaging involves the use of radiowaves of discern the location of tissues in the body. Pelvic ultrasounds are non-invasive and routine procedures. Because ultrasound imaging has a low contrast resolution, it cannot be used to differentiate between normal and malignant tissues, and is often used for determination of the general size of tissue structures.

In all of the above organ imaging techniques it should be apparent that the marker of the instant invention would be readily detectable as an opaque material in the body. As this marker is positioned with the cervix and uterus, it should be apparent that there is no need to administer any type of contrast material to very accurately determine the location of the cervix and uterus.

One of the primary reasons for accurately and easily determining the location of the cervix and uterus using the instant invention is to allow for the precise delivery of radiation therapy to the cervix and uterus. As discussed above, radiation therapy is one of the more effective treatments for cervical cancer. However, current radiation therapy of cervical and uterine cancer usually involves the use of internal radiation therapy, which is uncomfortable and unpleasant for the patient. Specific targeting of the cervix and uterus through the use of the instant invention will allow for the accurate and safe administration of IMRT, a highly effective procedure for treating cervical and uterine cancers that is much more tolerable for the patient.

To those experienced in the art, numerous modifications will be apparent within the scope of the invention as presented. This applies, for example, to the purpose of using the device for visualizing the cervix and uterus, as a medical practitioner might wish to locate these organs for purposes other than administering radiotherapy. The scope of the invention is set forth in the claims below.

What is claimed is:

1. A device for precisely locating the position of the cervix and uterus during imaging, comprising means opaque to radiation insertable in the cervix, and means for fixing said device with respect thereto.

2. The device of claim 1, wherein said fixing means includes sutures.

3. The device of claim 1, wherein said opaque means is a metallic insert.

4. A device for precisely locating the position of the cervix and uterus during imaging, comprising
a sleeve, and
a marker opaque to radiation held in place within the sleeve; wherein said device is adapted to be fixed to the cervix.

5. The device of claim 4, wherein the sleeve is fixed to the cervix by suturing the device to the cervix.

6. The device of claim 4, wherein said marker is held in place in said sleeve using an inert filling material.

7. The device of claim 6, wherein the inert filling material is vinyl polysiloxane.

8. The device of claim 6, wherein the inert filling material is Reprosil.

9. The device of claim 4, wherein the sleeve is a Smit sleeve.

10. The device of claim 4, wherein the marker is a solder wire.

11. A method for locating the cervix and uterus, comprising
fixing a device opaque to radiation through the cervix into the uterus, and
using a method of organ visualization to determine the location of the cervix and uterus.

12. The method of claim 11, wherein the device comprises, a sleeve, and
a marker opaque to radiation held in place within the sleeve.

13. The method of claims 11 or 12, wherein the method of organ visualization is computerized tomography.

14. The method of claims 11 or 12, wherein the method of organ visualization is fluoroscopy.

15. The method of claims 11 or 12, wherein the method of organ visualization is an x-ray.

16. The method of claims 11 or 12, wherein the method of organ visualization is Magnetic Resonance Imaging.

17. The method of claims 11 or 12, wherein the method of organ visualization is ultrasound.

18. The method of claims 11 or 12, wherein the locations of the cervix and uterus are visualized for the purpose of targeting radiation therapy.

19. The method of claim 18, wherein the radiation therapy is Intensity-Modulated Radiation Therapy.

20. A method of treating cervical and uterine tumors using external radiation, comprising:
inserting a device opaque to radiation through the cervix into the uterus,
securely fastening the device to the cervix to serve as a locating marker relative to various tissues and organs in and around the cervix and uterus,
visualizing the cervix and uterus by means of an imaging technique that locates the tumor relative to said marker, and
impinging external radiation on said tumor.
